(19) United States
(12) Patent Application Publication
(10) Pub. No.: US 2001/0037128 A1
(43) Pub. Date: Nov. 1, 2001

(54) Forward resector

(76) Inventor: Jared Arambula, San Diego, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/771,006

(22) Filed: Jan. 26, 2001

Related U.S. Application Data
(60) Provisional application No. 60/178,508, filed on Jan. 27, 2000.

(51) Int. Cl. A61M 29/00

(52) U.S. Cl. 606/198

(57) ABSTRACT

The present invention provides a system for resecting body tissues, and is especially useful in resecting body tissues in a cannulated approach into the patient. A tissue resector comprising a pair of curved cutting elements which are pivotally mounted on a distal end of a tubular member. The curve cutting elements are preferably shaped as sections of a hollow sphere which open and close to resect tissue with cutting elements articulating within the interior diameter of the tubular member. A novel system for resecting tissues wherein the cutting elements do not extend beyond the inner diameter of the tubular member to which they are attached which is inserted into the patient.

An actuation mechanism is provided for opening and closing the curved cutting elements (i.e., jaws). The actuation mechanism may comprise an inner tubular member which is slidably received within a main (outer) tubular member, and a linkage which joins the distal ends of the interior tubular member with each of the curved cutting elements. Movement of the inner tubular member within the outer tubular member causes the linkage to open and close the curved cutting elements.

### RELATED APPLICATION

The present application is a regular filing of, and claims the benefit of priority from U.S. Provisional Patent Application Ser. No. 60/178,508, filed Jan. 27, 2000, the full disclosure of which is incorporated herein by reference in its entirety for all purposes.

### TECHNICAL FIELD

The present invention is related to tissue resection systems.

### BACKGROUND OF THE INVENTION

Numerous systems have been developed for resecting tissues. Unfortunately, a limitation common to many of these systems is their bulkiness, which makes them poorly suited for use in cannulated minimally invasive surgical procedures. In addition, it has proven difficult to operate such systems through narrow cannulae as such resectors often have their cutting surfaces at their lateral edges (i.e.: sides) and are not adapted to cut in a forward direction (i.e.: straight ahead in a distal direction).

### SUMMARY OF THE INVENTION

The present invention provides a system for resecting body tissues, and is especially useful when resecting body tissues when used in a percutaneous cannulated approach into a patient. As will be explained, the present system is ideally suited to resect tissue in a “forward” direction (i.e.: in a straight line projecting distally along the axis of the device).

In a preferred aspect, the present tissue resector comprises a pair of curved cutting elements which are attached to a distal end of an outer member. Optionally, this outer member is tubular in cross section, and is hollow (to permit movement of an inner member therethrough), but the outer member need not be tubular in cross section.

The curved cutting elements act as jaws which are advanced distally into a tissue mass (by moving the resector forward) and then closed (by moving the inner member backward), thereby cutting away (i.e.: resecting) body tissue.

The curved cutting elements are preferably shaped as sections of a hollowed out sphere and are pivotally mounted to both the inner and outer tubular members. The inner tubular member is received within the outer tubular member.

The curved cutting elements can be opened and closed simultaneously to resect tissue with the curved cutting elements preferably articulating within dimensions not exceeding the interior diameter of the tubular member. As such, the present invention provides a novel system for resecting tissues wherein the pair of curved cutting elements does not extend beyond the inner diameter of the outer tubular member to which they are attached.

An actuation mechanism is provided for opening and closing the curved cutting elements (i.e., the “jaws”). In a preferred aspect, the actuation mechanism comprises the inner member which is slidably received within the outer tubular member, and a linkage which joins the distal ends of an interior tubular member with each of the curved cutting elements.

Longitudinal (i.e.: axial) movement of the inner member within the hollow outer tubular member causes the linkage to open and close the curved cutting elements.

The present system is advantageously suited to be inserted into the patient through an operating cannula, resecting tissue in a forward axial direction. In preferred aspects, the inner tubular member is connected to a suction line such that cut tissue may be removed from the operating area without having to remove the forward resector from the cannula.

### BRIEF DESCRIPTION OF THE DRAWINGS

FIG. 1 is a perspective view of the present invention.

FIGS. 2A is a side elevation view of the present invention in an open position, with interior components shown in dotted lines.

FIG. 2B is a side elevation view of the present invention in an open position, with the outer tubular member removed.

FIG. 3A is a side elevation view of the present invention in a closed position, with interior components shown in dotted lines.

FIG. 3B is a side elevation view of the present invention in a closed position, with the outer tubular member removed.

FIG. 4 is a perspective view of the present invention in a closed position, with the outer tubular member removed.

### DESCRIPTION OF THE BEST MODE OF THE PRESENT INVENTION

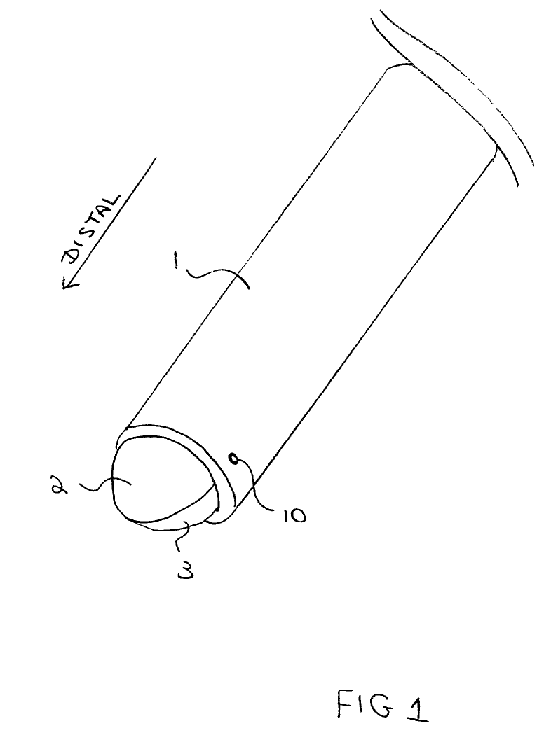

FIG. 1 shows a perspective view of present invention comprising an outer tubular member 1, an upper curved cutting element 2, a lower curved cutting element 3. Upper and lower cutting elements 2 and 3 are mounted to the distal end of outer tubular member 1 such that they pivot about pin 10.

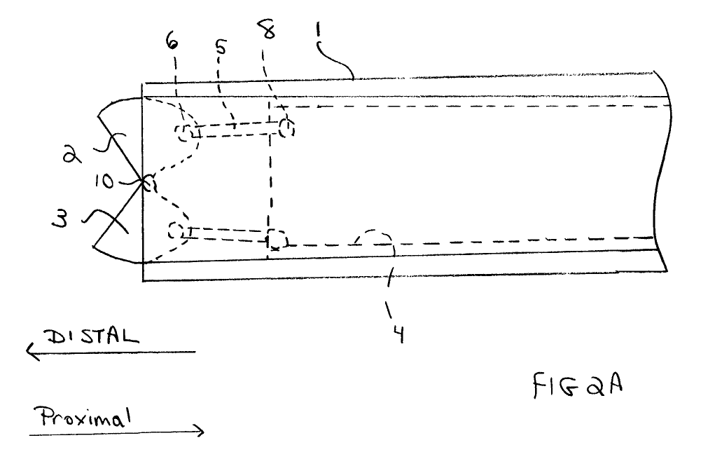

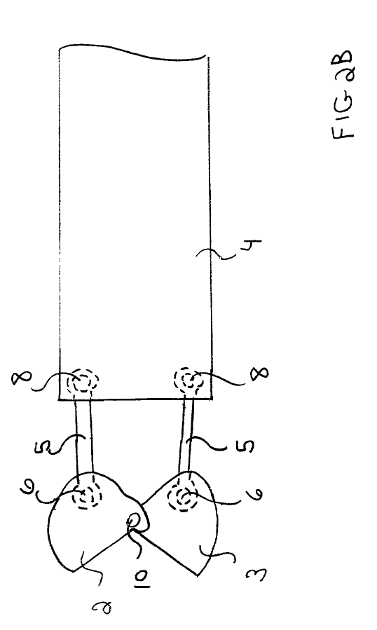

FIGS. 2A and 2B show the present forward resector with jaws 2 and 3 open. (i.e.: the position in which the resector is advanced into the tissue to be resected).

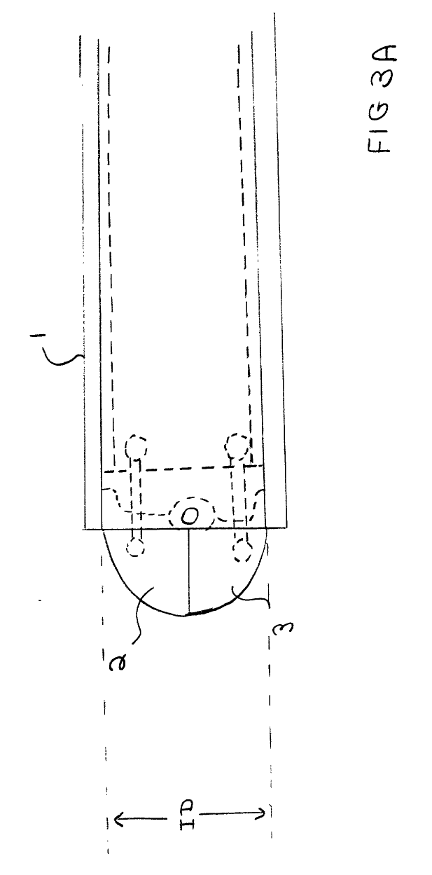

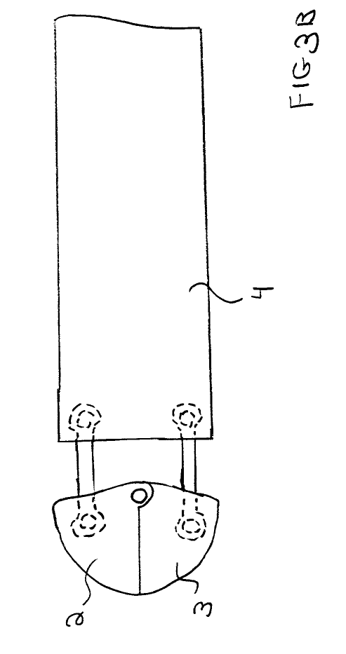

FIGS. 3A and 3B show the present forward resector with jaws 2 and 3 closed. (i.e.: the position in which the resector has cut away body tissue).

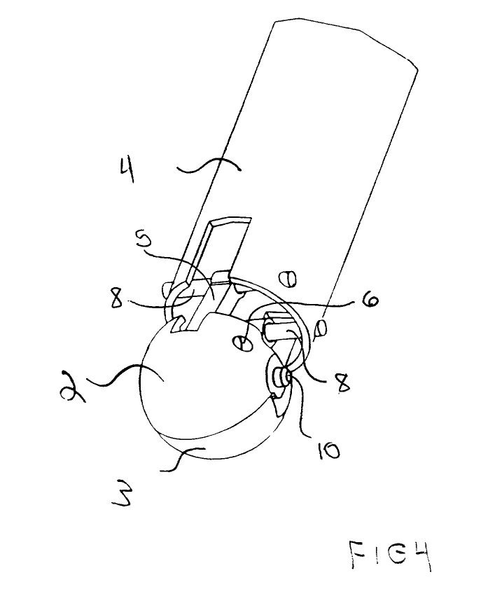

Jaws 2 and 3 are open and closed by movement of inner member 4 back and forth within outer tubular member 1. Specifically, an actuator arm 5, a jaw pin 6, and an inner tube pin 8 are provided. (This linkage can also be seen in FIG. 4) As inner member 4 is moved in a proximal direction (relative to outer member 1), inner tube pins 8 are also moved in a proximal direction (relative to outer member 1). However, jaws 2 and 3 also pivot about pin 10 (which is held at a fixed position at the distal end of outer member 1, causing jaws 2 and 3 to open. Conversely, as inner member 4 is moved in a distal direction (relative to outer member 1), inner tube pins 8 are also moved in a distal direction (relative to outer member 1). Therefore, since jaws 2 and 3 also pivot about pin 10 (which is held at a fixed position at the distal end of outer member 1), jaws 2 and 3 close, resecting tissue positioned therebetween.

An first important advantage of the present invention is that jaws 2 and 3 will resect tissue while at no times protruding radially outwards beyond the inner diameter ID (FIG. 3A) of outer member 1. A second important advantage of the present invention is that jaws 2 and 3 will simultaneously open and close when resecting tissue therebetween. Accordingly, the present invention provides an excellent “in-line” resector which can be introduced through a cannula with the tissue that is resected by the resector being that tissue which is disposed directly in front of the distal end of the resector.

In preferred aspects, inner member 4 may comprise a hollowed out tube which is connected to a suction line (not shown) such that cut tissue may be removed from the operating area without having to remove the forward resector from an operating cannula.

What is claimed is:

1. A tissue resector comprising:
an outer member;
a pair of curved cutting elements pivotally mounted opposite one another at the distal end of the outer member; wherein the curved cutting elements are shaped as sections of a sphere; and
an actuation mechanism for opening and closing the curved cutting elements.

2. The tissue resector of claim 1 wherein, each of the curved cutting elements is shaped as a section of a hollow sphere.

3. The tissue resector of claim 1 wherein, the actuator mechanism comprises:
an inner member slidably received within the outer member,
a linkage joining the distal end of the inner member with each of the curved cutting elements, wherein movement of the inner member within the outer member causes the linkage to open and close the curved cutting elements.

4. The tissue resector of claim 1 wherein, the curved cutting elements open and close within the interior diameter of the outer member.

5. The tissue resector of claim 3 wherein, the linkage comprises:
a longitudinal member which is pivotally attached to one of the curved cutting elements at one end, and pivotally attached to the distal end of the inner member at an opposite end.

6. The tissue resector of claim 3 wherein, the linkage comprises:
a first pin connected to a curved cutting element;
a second pin connected to the distal end of the inner member; and
a longitudinally member pivotally connected at opposite ends to each of the first and second pins.

7. A method of dissecting tissue comprising:
advancing the distal end of a member adjacent tissue to be resected; and
closing a pair of cutting elements mounted at the distal end of the member thereby resecting the tissue, wherein a pair of curved cutting elements are each shaped as sections of a hollow sphere.

8. The method of resecting tissue of claim 7 wherein, closing a pair of curved cutting elements comprises longitudinally displacing an actuator relative to the member, wherein the actuator is connected to said pair of cutting elements, and wherein longitudinal movement of the actuator causes angular movement of the cutting elements.

9. The tissue resector of claim 1, further comprising:
a suction line connected to the inner member for removing cut tissue from an operating area.
